(19) United States
(12) Patent Application Publication
(10) Pub. No.: US 2011/0092889 A1
(43) Pub. Date: Apr. 21, 2011

(54) THERAPEUTIC DEVICE

(76) Inventors: Carl Daniels, Rockhampton (AU); Nick Daniels, Rockhampton (AU); Wayne Daniels, Rockhampton (AU); Anthony Scott Horstman, Maylands (AU)

(21) Appl. No.: 12/735,745

(22) PCT Filed: Feb. 12, 2009
(86) PCT No.: PCT/AU2009/000165

§ 371 Date: Oct. 27, 2010

(30) Foreign Application Priority Data
Feb. 14, 2008 (AU) 2008900693

(51) Int. Cl. A61B 17/50; A61B 19/02; A61M 31/00

(52) U.S. Cl. 604/22; 606/162; 206/438

(57) ABSTRACT

A device for treating a passage in a body part, said device having a flexible elongate body comprising a leading segment that is inserted into an opening to the passage and passed therethrough until it exits the passage and a cleaning segment that follows the leading segment through the passage and debrides the passage as it is forced therethrough.

### TECHNICAL FIELD

This invention relates to a therapeutic device for treating a passage in a body. The invention has particular application to a therapeutic device used for cleaning a piercing and where desired applying a therapeutic substance or substances to tissue adjacent the piercing.

### BACKGROUND ART

The piercing of body parts has become commonplace. Piercing of the ear, eyebrow, tongue, navel and nose, to name but a few parts of the body, is generally accepted. A piercing, which is a passage formed by a sharp instrument, is essentially a wound. Like all wounds, piercings are susceptible to infection.

Body piercings are generally difficult to clean and consequently once an infection has taken hold it can be difficult to overcome. An infection of a piercing of the upper ear cartilage, for example, is especially serious. Treatment of the infection with an oral antibiotic preparation, is generally ineffective as cartilage doesn't have its own blood supply. This means that the drug cannot reach the site of infection. Infection of this kind can lead to cartilage damage and permanent ear deformity.

The removal of foreign matter and the application of anti-microbial preparations at the piercing site helps to reduce the incidence of infection. The prior art describes a number of devices or systems for cleaning and/or applying a therapeutic substance to a piercing. U.S. Pat. Nos. 4,497,402 and 6,146,398 are examples of such devices or systems.

U.S. Pat. No. 4,497,402 (Karos) describes an apparatus for cleaning and sterilizing a passage in an ear lobe, the apparatus comprising a string of absorbent material having a firm tip attached thereto. The apparatus is maintained in a sealed package adjacent a pad of absorbent material saturated with an antiseptic material. When cleaning of a piercing is desired the package is opened and the apparatus is inserted into and subsequently drawn through the piercing, the string acting both to debride and apply the antiseptic material.

U.S. Pat. No. 6,146,398 (Satterfield) discloses a tool for cleaning and applying medication to a piercing, the tool comprising a semi-rigid elongated member having a first section that cleans the piercing and a second section that has a medication absorbent layer. The cleaning section comprises a plurality of flexible, fibrous arms that help debride the piercing.

It is an aim of the invention to provide a therapeutic device that can prevent or treat an infected passage, such as a piercing, and has advantages over the prior art, particularly in the provision of a novel structure that promotes rigorous debridement of the piercing and concomitant application of a therapeutic substance.

### SUMMARY OF THE INVENTION

According to a first embodiment of the invention, there is provided a device for treating a passage in a body part, said device having a flexible elongate body comprising:

a leading segment that is inserted into an opening to the passage and passed therethrough until it exits the passage; and

a cleaning segment that follows the leading segment through the passage and debrides the passage as it is forced therethrough.

With respect to the first embodiment of the invention, the leading segment preferably exhibits greater rigidity than the cleaning segment. It will be appreciated by one of skill in the art that a semi-rigid or rigid leading segment aids insertion of the leading segment into the passage and provides the user with greater control as the leading segment is passed through the passage.

In a preferred embodiment, the leading segment is coated with a material where that imparts greater rigidity to said leading segment. The material may be any suitable substance. This may include, but is not limited to, a plastic material. The material may also increase the visibility of the leading segment and/or indicate a region that is to be handled. For example it may be coloured. It will be appreciated that a suitably coloured leading segment may provide greater contrast with the skin, thereby aiding the operator when inserting the leading segment into the passage. In other embodiments, the leading segment can be treated with a hardening agent.

Preferably, a forward end of the leading segment is bulbous or rounded. It will be appreciated by one of skill in the art that the forward end of the leading segment having a bulbous or rounded end aids insertion of the leading segment into the passage.

The cleaning segment may include a deformable circumferential surface. It is also preferable that at least the peripheral portion of the cleaning segment substantially along its length is adapted to deform as it passes through the passage. Preferably, the circumferential surface has a plurality of spaced apart ridges and valleys to assist in cleaning the passage. In some embodiments, the cleaning segment is formed from a single elongate member arranged into a series of connected elements that move between a rest position, at which position the elements are adjacent, to an extended position, at which position the elements are separated.

It will be appreciated that when the cleaning segment is drawn through the passage, the connected elements move from the rest to the extended position. It will also be appreciated that when drawn through the passage, the connected elements resemble, in profile, a plurality of spaced apart ridges and valleys, wherein at least one ridge of a connected element contacts a wall of the passage thereby removing dead or infected tissue.

In a particularly preferred embodiment, the cleaning segment is or includes a coil. It will be appreciated that the that helical member forming the coil may be arcuate, triangular, rectangular or any other suitable cross-sectional shape.

Preferably, the cleaning segment is shaped such that, when at the extended position, at least one surface of a portion of the cleaning segment contacts a wall of the passage. The cleaning segment may have, but is not limited to, an arcuate, triangular or rectangular cross-sectional shape.

Preferably, an outer surface of the cleaning member is abrasive. In a preferred embodiment, the cleaning segment is coated in an abrasive substance. In other embodiments, the outer surface is irregular having either corrugations or protrusions.

Preferably, the device is at least partially coated or impregnated with at least one therapeutic substance. Alternatively the therapeutic substance may be compounded with the material used to form the device.

In a preferred embodiment, the cleaning segment includes at least one therapeutic or cleaning substance. It will be appreciated that the at least one therapeutic substance may prevent or treat an infection of the passage or promote healing of the passage. In other embodiments, the cleaning segment comprises one or more therapeutic substances that act to prevent or treat an infection and heal the passage. A therapeutic substance may be, but is not limited to, an astringent, antiseptic, antibiotic, exfoliate, growth factor or steroid or a synergistic mixture of substances.

Preferably, the leading segment is connected to the cleaning segment by a transition segment, wherein the transition segment comprises a radius or curve such that the motion of the leading segment through the passage is smoothly transferred to the cleaning segment without sharply angled portions or edges between the two segments that could catch on a body part and cause pain or injury.

The device can be manufactured from any suitable material but is typically manufactured from metals or plastics, the latter being preferred. Plastics may include, but are not limited to, polyurethane or nylon. In a particularly preferred embodiment, the device is manufactured from a single thermally formable plastic rod that is shaped to form the segments of the device.

The device may be reusable or single use. When the device is reusable, it is preferably manufactured from an autoclavable or sterilisable material.

It will be appreciated by one of skill in the art that the dimensions of the device are based upon the diameter of a passage to be treated. For example, items such as earrings require a passage having a diameter of approximately 0.5 to 1 mm, whereas larger ornamental jewelry such as barbells, require a passage having a diameter greater than or approximately equal to 1 mm. Accordingly, the diameter of the body of the device preferably approximates that of the passage to be treated.

In a particularly preferred embodiment, the device of the invention is formed from a unitary thermally formable body, having:

a leading segment with greater rigidity than the remainder and that is inserted into an opening to the passage and passed therethrough until it exits the passage;

a cleaning coil segment that follows the leading segment through the passage, such that when the coil enters the passage, adjacent portions of the coil are deformed providing at least one abrasive surface capable of debriding the passage; and

a transition segment located between the leading segment and the cleaning segment and which is curved such that the motion of the leading segment through the passage is evenly transferred to the cleaning segment.

According to a second embodiment of the invention, there is provided a package comprising a plurality of therapeutic devices for treating a passage in a body, wherein said package comprises a holder that retains the plurality of elongate therapeutic devices, and wherein said the package includes a resilient portion with a number of openings therein each adapted to receive at least a portion of an elongate therapeutic device, and a container having a containing portion and a neck portion, the neck portion dimensioned to receive and retain the resilient portion holding the elongate therapeutic devices.

With respect to the second embodiment of the invention, the holder preferably comprises:

a flexible member comprising a plurality of spaced apart recesses that each removably retain a therapeutic device; and

a container that removably receives the flexible member.

The flexible member preferably forms a strip, wherein said plurality of spaced apart recesses are located substantially perpendicular to its length. The strip may be manufactured from any suitable material but is preferably manufactured from a resilient material such as foam. In some embodiments, the strip is manufactured from a material that can absorb an aqueous solution or retain a desiccant or powder. A recess may be formed during the manufacture of a flexible member or may be formed in a pre-existing material, for example, by making an incision in a flexible foam member.

Preferably, each device is removably retained by a recess formed in the strip such that a portion of the leading segment extends from the member and the cleaning segment extends from an opposing portion of the member. In a preferred embodiment, the recesses are arrayed along the length of the strip such that the distance separating adjacent recesses gradually increases. It will be appreciated that a user typically removes a device from the strip by grasping the leading portion and pulling such that the remaining portions of the device, including particularly the cleaning portion, are drawn through the recess. When the device comprises a cleaning portion that is a coil, removal of the device results in the extension of the coil as it moves through the recess.

Preferably, the strip comprising said plurality of devices is rolled into a bundle. In a preferred embodiment, the bundle is prevented from unfurling by a tie. The tie may be, but is not limited to, a tape that is wrapped around the periphery of the bundle when said bundle reaches a desirable dimension. In a particularly preferred embodiment, the tape is furnished with indicia providing directions of use.

In other embodiments, the strip retains at least one therapeutic or cleaning substance. It will be appreciated that when the strip is manufactured from an absorbent material at least one therapeutic or cleaning substance may be retained by the strip. For example, the strip may be soaked in an aqueous solution comprising the at least one therapeutic or cleaning substance. It will also be appreciated that when a device is removed from a recess that is formed in a strip that retains at least one therapeutic or cleaning substance, the at least one substance is applied to the cleaning segment of the device. It will further be appreciated that when the cleaning segment is a coil, removal of the device results in the extension of the coil allowing substantially uniform application of the at least one therapeutic substance to the surface of the coil.

The container preferably comprises an opening, a neck, and a reservoir, wherein the neck releasably retains the bundle. In a preferred embodiment, the neck of the container comprises intrusions that help to orient and retain the bundle within the neck. The container may also comprise a lid. A device retained by the strip and a part of said bundle is typically oriented such that a portion of the leading segment extends from the bundle towards an opening in the container and the cleaning segment extends from an opposing portion of the member towards the reservoir. In a preferred embodiment, the reservoir of the container retains at least one therapeutic substance. It will be appreciated that a cleaning segment of a device retained in the bundle may be immersed in an aqueous solution comprising the at least one therapeutic substance.

In order that the invention may be more readily understood and put into practice, one or more preferred embodiments thereof will now be described, by way of example only, with reference to the accompanying drawings.

### BRIEF DESCRIPTION OF THE DRAWINGS

FIG. 1 provides a side view of a therapeutic device according to the invention.

FIG. 2 provides a view of a therapeutic device according to the invention in situ.

FIG. 3 provides a magnified view of the cleaning segment according to the invention.

FIG. 4 provides a side view of a package according to the invention.

FIG. 5 provides a perspective view of a strip according to the invention.

FIG. 6 provides a perspective view of a tied bundle according to the invention.

### DESCRIPTION OF PREFERRED EMBODIMENTS

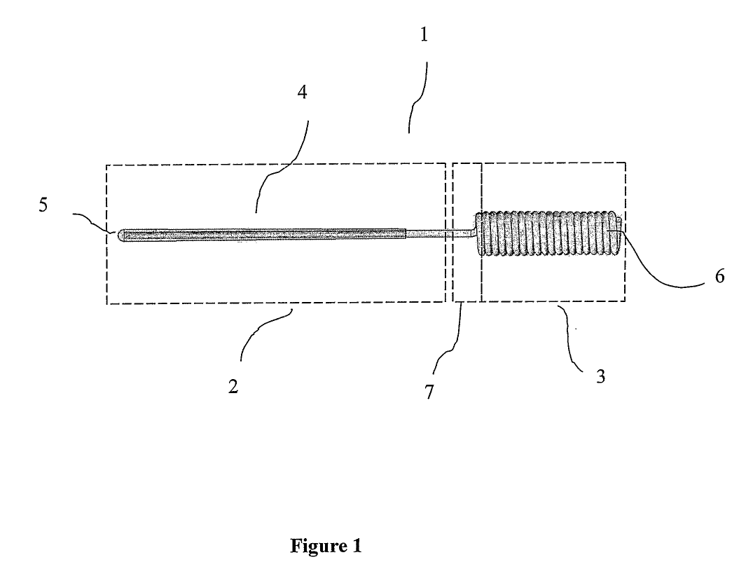

In FIG. 1 there is shown a therapeutic device having a flexible elongated body 1 comprising a leading segment 2 and a cleaning segment 3. The leading segment 2 is coated with a second material 4 that imparts greater rigidity to the leading segment relative to the remainder of the device. An end 5 of the leading segment is rounded. The cleaning segment 3 is arranged into a series of adjacent arcuate connected members forming a coil 6. The leading segment is connected to the cleaning segment by a transition segment 7, which incorporates a curve such that the motion of the leader through the passage is smoothly transferred to the coil 6 without excessive discomfort to the recipient.

It will be appreciated that when the cleaning segment of the device is a coil, the coil may have an outer diameter of approximately 4 mm and a length of approximately 12 mm, providing a fully extended length of approximately 120 mm. The coil is typically constructed from a flexible thermoplastic material, formed and biased to retain the coiled shape. The leading segment typically has a diameter of approximately 0.5 to 0.8 mm and length of approximately 30 mm for most applications.

It will also be appreciated that the end of the leading segment opposite the cleaning segment may be cut or otherwise treated to provide a suitable end shape to accept a second material that is applied to form a rounded or bulbous finished shape. In the preferred embodiment, the rounded or bulbous end of the leading segment is cut to produce a flat end that is then dipped into a low-viscosity material and rapidly cured by heat or radiation as it begins to flow toward the leading segment. A bulbous end may have a diameter of approximately 0.8 to 1.2 mm.

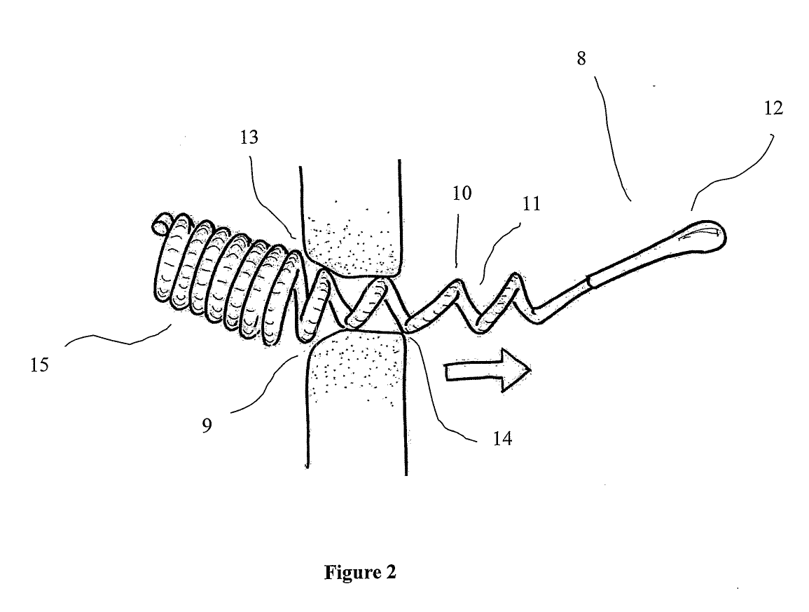

In FIG. 2 there is shown a therapeutic device 8 as it is drawn through a passage 9. The arrow denotes the direction in which the device is pulled and/or passed through the passage. As shown, the connected elements comprising the cleaning segment resemble, in profile, a plurality of spaced ridges 10 and valleys 11, wherein at least one ridge of a connected element contacts a wall of the passage thereby removing dead or infected tissue. In operation, the leading segment 12 is inserted into an opening 13 of the passage 9 and passed therethrough until it protrudes from the exit 14, at which position the leading segment is grasped by the operator and the coiled cleaning segment 15 is drawn through the passage. It will be appreciated that when the cleaning portion is a coil the cleaning action of the device is characterised by a reaming motion of the external surfaces of the coil as it extends through the passage. It will also be appreciated by one of skill in the art that the relative “tightness” of the passage will induce a resistance to the pull, making the coil extend within the passage, with the reduced effective diameter of the coil defined by the balance of the passage diameter and the friction provided by a wall of the passage. In other words, the spring will extend further in small diameter passages resulting in a similar pull-through force as a shorter extension in a larger diameter passage. Thus, the cleaning action of the spring may be considered reasonably standard across various passage diameters.

It will further be appreciated that as the tail end of the coil exits the hole, the spring moves the connected elements making up the coil move from the extended position towards the rest position. The force of this relaxation is not great, and does not provide a reaction sufficient to eject material or cause alarm to the user.

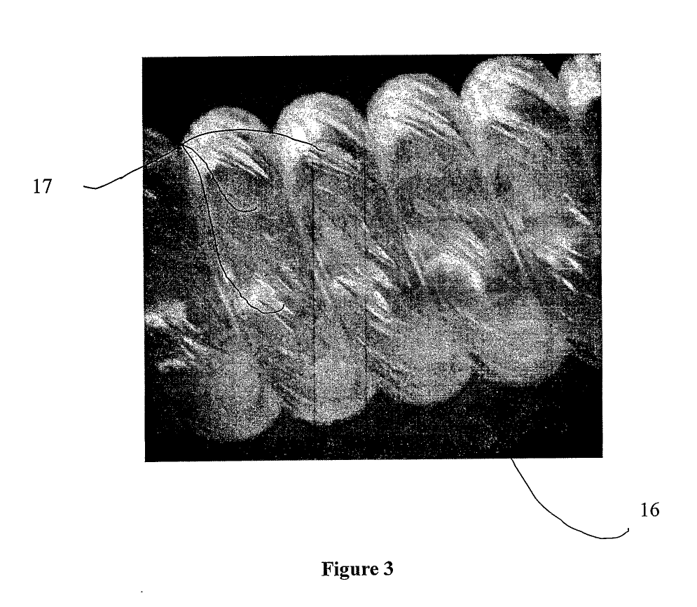

FIG. 3 shows a magnified view of a cleaning segment 16 according to the invention in which the cleaning segment is arranged to form a coil, the external surface of which comprises a plurality of corrugations 17. It will be appreciated by one of skill in the art that such corrugations provide an abrasive surface with which to debride the passage.

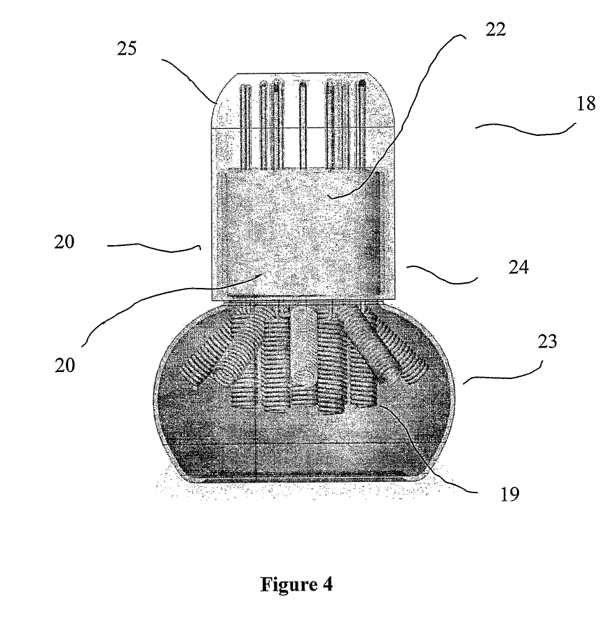

In FIG. 4 there is shown a package 18 comprising a plurality of therapeutic devices according to the invention 19. The package includes a holder that releasably retains said plurality of devices. The holder comprises: a flexible strip 21 that is rolled to form a bundle 22; and a container 23 having a neck 24 that releasably retains the bundle 22. The package also includes a cover 25 that is releasably retained by the container. It will be appreciated that the cap protects the therapeutic devices from contamination. It will be appreciated that therapeutic device according to the invention are extracted from the bottle by selecting and pulling the portion of the leading segment extending from the edge of the strip. As the operator pulls the leading segment the cleaning segment that is a coil may tangle. To alleviate this, instructions are provided on the package or other location to direct the user to rotate the bottle and leading segment of the device in opposing directions.

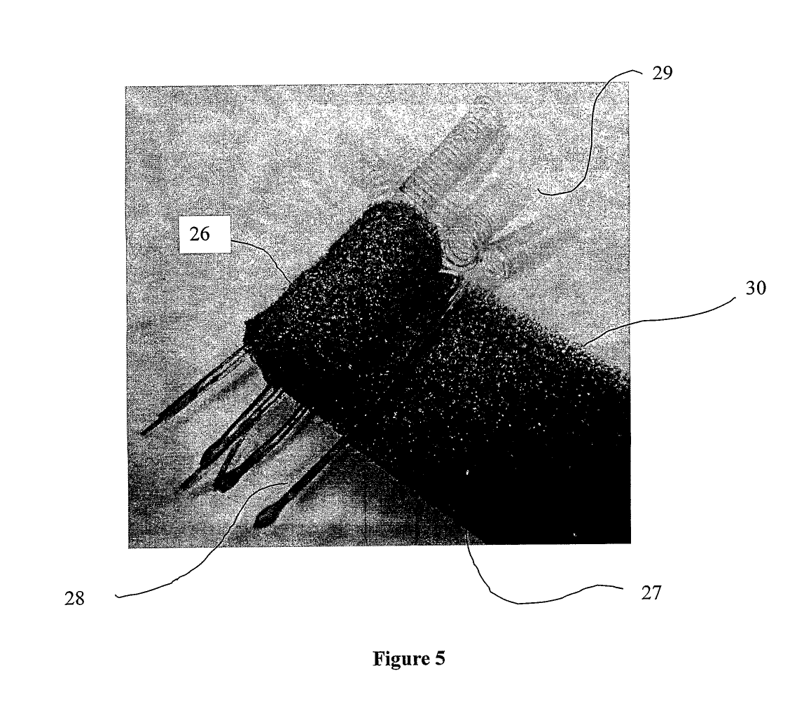

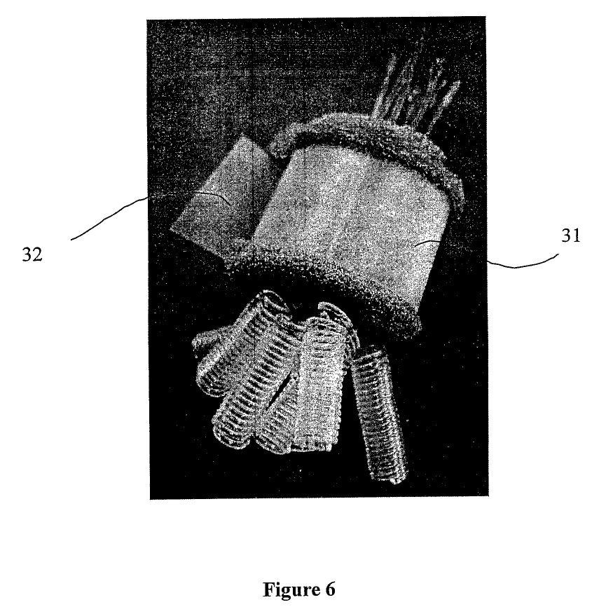

In FIG. 5 there is shown an semi-open foam strip 26 comprising spaced apart recesses or slits arranged along its length. A slit 27 releasably retains a device according to the invention such that a portion of the leading segment 28 extends from the edge of the strip and the cleaning segment 29 extends from an opposing edge 30 of the strip. Devices according to the invention are arranged side-by-side, and the strip is then rolled up. The open-cell foam strip shown in FIG. 6 is approximately 20 mm wide and has an uncompressed depth of 5 mm. The slits are typically made to a depth of approximately half the thickness of the leading segment. It will be appreciated that the slits may be spaced closer together at one end. It will also be appreciated that at least one therapeutic substance may be retained by the strip wherein the at least one therapeutic substance is applied to the cleaning segment as it is drawn through the strip.

In FIG. 6 there is shown a bundle 31 secured by tie that is an adhesive tape 32. The adhesive tape is wrapped around the roll as it reaches the desired finished diameter thereby fixing the strip in the rolled state. The bundle of therapeutic devices is then placed and is releasably retained by the neck of the container.

The foregoing embodiments are illustrative only of the principles of the invention, and various modifications and changes will readily occur to those skilled in the art. The invention is capable of being practiced and carried out in various ways and in other embodiments. It is also to be understood that the terminology employed herein is for the purpose of description and should not be regarded as limiting.

Mere reference to background art herein should not be construed as an admission that such art constitutes common general knowledge or prior art in relation to this application. The term “comprise” and variants of the term such as “comprises” or “comprising” are used herein to denote the inclusion of a stated integer or stated integers but not to exclude any other integer or any other integers, unless in the context or usage an exclusive interpretation of the term is required.

1. A device for treating a passage in a body part, said device having a flexible elongate body comprising a leading segment that is inserted into an opening to the passage and passed therethrough until it exits the passage and a cleaning segment that follows the leading segment through the passage and debrides the passage as it is forced therethrough.

2. The device for treating a passage in a body part as claimed in claim 1 wherein the leading segment has greater rigidity than the cleaning segment.

3. The device for treating a passage in a body part as claimed in claim 1 wherein the leading segment is coated with a material that imparts greater rigidity to said leading segment.

4. The device for treating a passage in a body part as claimed in claim 1 wherein a forward end of the leading segment is bulbous or rounded.

5. The device for treating a passage in a body part as claimed in claim 1 wherein the cleaning segment includes a deformable circumferential surface.

6. The device for treating a passage in a body part as claimed in claim 1 wherein the cleaning segment has a helical shape.

7. The device for treating a passage in a body part as claimed in claim 1 wherein the leading segment is connected to the cleaning segment by a transition segment, wherein the transition segment comprises a radius or curve such that the motion of the leading segment through the passage is smoothly transferred to the cleaning segment without sharply angled portions or edges between the two segments that could catch on a body part and cause pain or injury.

8. The device for treating a passage in a body part as claimed in claim 1 wherein the device is manufactured from a single thermally formable plastic rod that is shaped to form the segments of the device.

9. The device for treating a passage in a body part as claimed in claim 5 wherein the cleaning segment is formed from a single elongate member arranged into a series of connected elements that move between a rest position, at which position the elements are adjacent, to an extended position, at which position the elements are separated wherein as the cleaning segment is drawn through the passage, the connected elements move from the rest to the extended position.

10. The device for treating a passage in a body part as claimed in claim 9 wherein the cleaning segment is shaped such that, when at the extended position, at least one surface of a portion of the cleaning segment contacts a wall of the passage.

11. The device for treating a passage in a body part as claimed in claim 1 wherein an outer surface of the cleaning member is abrasive.

12. The device for treating a passage in a body part as claimed in claim 1 wherein at least the cleaning portion of the device is at least partially coated or impregnated with at least one therapeutic or cleaning substance.

13. A unitary thermally formable body for treating a passage in a body part, having a leading segment with greater rigidity than the remainder and that is inserted into an opening to the passage and passed therethrough until it exits the passage; a cleaning coil segment that follows the leading segment through the passage, such that when the coil enters the passage, adjacent portions of the coil are deformed providing at least one abrasive surface capable of debriding the passage; and a transition segment located between the leading segment and the cleaning segment and which is curved such that the motion of the leading segment through the passage is evenly transferred to the cleaning segment.

14. A package comprising a plurality of elongate therapeutic devices for treating a passage in a, wherein said package comprises a holder that retains the plurality of elongate therapeutic devices, and wherein said package includes a resilient portion with a number of openings therein each adapted to receive at least a portion of an elongate therapeutic device, and a container having a containing portion and a neck portion, the neck portion dimensioned to receive and retain the resilient portion holding the elongate therapeutic devices.

15. The package as claimed in claim 14 wherein the holder comprises a flexible member comprising a plurality of spaced apart recesses that each removable retain an elongate therapeutic device and a container that removably receives the flexible member.

16. The package as claimed in claim 14 wherein the flexible member is a strip, wherein said plurality of spaced apart recesses are located substantially perpendicular to its length.

17. The package as claimed in claim 15 wherein each elongate therapeutic device is removable retained by a recess formed in the strip such that a portion of the leading segment extends from the member and the cleaning segment extends from an opposing portion of the member.

18. The package as claimed in claim 17 wherein an elongate therapeutic device provided with a leading portion and a cleaning portion is removed from the strip by grasping the leading portion and pulling such that the remaining portions of the device, including particularly the cleaning portion, are drawn through the recess.

19. The package as claimed in claim 18 wherein the strip retains at least one therapeutic or cleaning substance.

20. The package as claimed in claim 14 wherein the container comprises an opening, a neck, and a reservoir, wherein the neck releasably retains the bundle.
